(19) United States
(12) Patent Application Publication
(10) Pub. No.: US 2010/0222421 A1
(43) Pub. Date: Sep. 2, 2010

(54) Usage of Baicalein in the Preparation of a Pharmaceutical Composition for the Treatment of Traumatic Brain Injury

(75) Inventors: Jia-Yi WANG, Taipei City (TW); Wen-Hsin HUANG, Taipei City (TW); Szu-Fu CHEN, Taipei City (TW)

Correspondence Address: SCHMEISER OLSEN & WATTS, 18 E UNIVERSITY DRIVE, SUITE # 101, MESA, AZ, 85201, US

(73) Assignee: National Defense Medical Center, Taipei (TW)

(21) Appl. No.: 12/395,287

(22) Filed: Feb. 27, 2009

(51) Int. Cl. A61K 31/352; A61P 25/00

(52) U.S. Cl. 514/456

(57) ABSTRACT

The invention provides a novel medical use of Baicalein, and in particular, a use of baicalein in the preparation of a pharmaceutical composition useful for treating traumatic brain injury. The baicalein pharmaceutical composition in this invention includes a treating effective amount of baicalein and a suitable pharmaceutically acceptable excipient or carrier. The baicalein pharmaceutical composition is applicable for improving the behaviour function deficit in traumatic brain injury, reducing the contusion volume of traumatic brain injury, improving the brain neuronal degeneration in traumatic brain injury, reducing proinflammatory cytokines such as tumor necrosis factor-α (TNF-α), interleukin-1β (IL-1β), interleukin-6 (IL-6) or other relative inflammation factor induced in traumatic brain injury.

### BACKGROUND OF THE INVENTION

1. Field of the Invention

The invention relates to a novel medical use of baicalein, and in particular, to a pharmaceutical composition containing baicalein used for treating traumatic brain injury, and to a field of treating traumatic brain injury.

2. Description of the Prior Art

Causes of traumatic brain injury include traffic accidents, crashing from high places, sport or leisure activities. In Taiwan, traumatic brain injury is often happened in traffic accidents, especially when motorcycles are involved, and causes high mortality. Even the subject might survive; about 25% of the subject would become paralyzed. According to the statistics by the Department of Health in Taiwan, the number of death caused by traffic accidents involving motorcycles from 2001 to 2003 had been decreased remarkably since the government enforced the regulation of wearing safety helmets, and the statistics indicated that death number have been decreased year by year ever since. Unfortunately, traumatic brain injury remains an important social and healthy problem in that serious traumatic brain injury may cause even up to 57% of subjects having bad prognosis or neurological sequela, thereby brings about familial, social and economic burdens. Although traumatic brain injury has generally been considered as a problem treated by a neurosurgeon, 10-20% of the subject needs to be treated by surgery according to statistical survey. Furthermore, traumatic brain injury is a dynamic process in that, within a period shortly after the occurrence of a traumatic brain injury, proinflammatory cytokines such as, tumor necrosis factor-α (TNF-α), interleukin-1β (IL-1β), interleukin-6 (IL-6) or other relative inflammatory factors may increase greatly so that secondary injury such as death of brain neuronal cells might be induced. Moreover, harmful radical products might increase, thereby resulted in the oxidative damage on cells of brain tissue and affected seriously further the behaviour function of animal or human bodies. Currently, neurological medicine still lacks a fast and immediate effective approach to treat secondary injury occurred after a traumatic brain injury.

Baicalein is a major flavonoid extracted from the root of Scutellaria baicalensis Georgi. (also called Huang Qin), a traditional oriental medicine widely used in treating allergic and inflammatory diseases. This flavonoid has proved its potent anti-inflammatory effects in vitro as well as in vivo (Huang et al., 2006). In this regard, baicalein has been reported to scavenge free radicals (Huang et al., 2006; Wu et al., 2006) and inhibit lipid peroxidation activities (Huang et al., 2006; Wu et al., 2006). However, no information is available concerning the possible therapeutic efficacy of baicalein administered after acute traumatic brain injury (TBI).

Controlled cortical impact (CCI) is a readily reproducible model of TBI and displays many of the same pathophysiological hallmarks, such as oedema formation, motor and cognitive behavioural impairments and necrotic and apoptotic cell death (Morales et al., 2005).

The aim of this invention was to evaluate the effect of baicalein in TBI, based on the hypothesis that post-injury baicalein treatment would reduce functional deficits and extent of anatomical brain damage. In addition, baicalein would attenuate levels of proinflammatory cytokines TNF-α, IL-1β and IL-6 after controlled cortical impact (CCI) injury.

With respect to the above-described problem and hypothesis, the invention provides a novel medical use of baicalein in the preparation of a pharmaceutical composition that can effectively treat traumatic brain injury, wherein said pharmaceutical composition can be administrated immediately after traumatic brain injury to reduce proinflammatory cytokines such as tumor necrosis factor-α (TNF-α), interleukin-1β (IL-1β), interleukin-6 (IL-6) or other relative inflammatory factors, to improve the secondary injury such as nerve cell death and the like after traumatic brain injury, and to improve the deficit of Behaviour function in traumatic brain injury.

This invention had been published on British Journal of Pharmacology (BJP) on Sep. 8, 2008 and titled as “Post-injury baicalein improves histological and functional outcomes and reduces inflammatory cytokines after experimental traumatic brain injury.”

### SUMMARY OF THE INVENTION

The primary object of the invention is to provide a novel medical use of baicalein in the preparation of a pharmaceutical composition that can effectively treat traumatic brain injury, wherein said pharmaceutical composition is applicable for treating traumatic brain injury, and can achieve an object of improving the Behaviour function deficit in traumatic brain injury.

The secondary object of the invention is to provide a novel medical use of baicalein in the preparation of a pharmaceutical composition useful for treating traumatic brain injury, wherein said pharmaceutical composition can be administrated immediately after traumatic brain injury, to reduce the volume of traumatic brain injury, and achieve an object of improving the Behaviour function deficit in traumatic brain injury.

Another object of the invention is to provide a novel medical use of baicalein in the preparation of a pharmaceutical composition useful for treating traumatic brain injury, wherein said pharmaceutical composition can be administered immediately after traumatic brain injury to achieve the object of reducing proinflammatory cytokines in traumatic brain injury.

In addition, another object of the invention is to provide a novel medical use of baicalein in the preparation of a pharmaceutical composition useful for treating traumatic brain injury, wherein said pharmaceutical composition can be administrated immediately after traumatic brain injury to achieve an object of improving secondary injury such as brain neuronal degeneration in traumatic brain injury.

The novel medical use of baicalein in the preparation of a pharmaceutical composition useful for treating traumatic brain injury that can achieve the above-described objects of the invention comprises:

use of baicalein or a pharmaceutical composition containing the same in treatment of traumatic brain injury and the secondary injury thereof, wherein said baicalein or a pharmaceutical composition containing the same is administrated immediately after traumatic brain injury so as to reduce the volume of traumatic brain injury and improve the Behaviour function deficit and other secondary injury caused by traumatic brain injury, or to reduce proinflammatory cytokines induced by traumatic brain injury; wherein said pharmaceutical composition containing baicalein can contain further suitable pharmaceutically acceptable excipients or carriers to form a pharmaceutical composition containing baicalein useful for treating traumatic brain injury or secondary injury thereof;

wherein the well-known structure of said baicalein is shown in FIG. 8.

In addition to the novel medical use of baicalein or a pharmaceutical composition containing the same in the treatment of traumatic brain injury, the invention provides further a pharmaceutical composition containing baicalein for treating traumatic brain injury or secondary injury thereof.

Also, in this invention, neurological status in animals suffering injuries from the Controlled cortical impact (CCI) was evaluated using the rotarod, tactile adhesive removal, modified neurological severity scores (mNSS) and beam walk tests. Both mRNA expression and protein levels of the cytokines were assessed through the use of real time quantitative reverse transcriptase-PCR (RT-PCR), ELISA and immunohistochemistry. And the results suggest that post-injury bacalein can reduce cytokine upregulation and ameliorate the extent of injury associated with CCI.

These features and advantages of the present invention will be fully understood and appreciated from the following detailed description of the accompanying drawings.

### BRIEF DESCRIPTION OF THE DRAWINGS

FIGS. 1A-D show the effects of single-dose and multiple-dose baicalein treatment on functional outcomes in rats with controlled cortical impact (CCI) injury, as evaluated by (a) rotarod, (b) tactile adhesive removal, (c) modified neurological severity score (mNSS) and (d) beam walk tests. CCI injury impaired performance on all tests in vehicle-treated and baicalein-treated injured rats. As shown in FIG. 1A, both the single-dose and multiple-dose baicalein-treated rats showed significantly better performance on the rotarod test than vehicle-treated rats did between 1 and 28 days after injury. Compared with single-dose baicalein, multiple-dose baicalein resulted in a significantly better rotarod performance on days 1, 4, 7 and 21. As shown in FIG. 1B, compared with vehicle-treated rats, both the single-dose and multiple-dose baicalein-treated rats had significantly lower adhesive-removal times on days 1, 4 and 7. The adhesive removal time was significantly lower only on day 7 in rats treated with multiple doses of baicalein comparing with rats treated with a single dose of baicalein. As shown in FIG. 1C, the mNSS scores were significantly lower for the single-dose baicalein-treated group than vehicle-treated group on days 1 to 14 and significantly lower for the multiple-dose group than the vehicle-treated group on days 1 to 28. As compared with a single-dose baicalein treatment, multiple-dose treatment significantly reduced neurological deficits on mNSS on days 4 and 7. As shown in FIG. 1D, the hindlimb motor scores were significantly decreased when compared to those of the vehicle group on days 4, 7, 14 and 21 for the single-dose baicalein-treated group and on days 1, 4, 14, 21 and 28 for the multiple-dose group. There were no significant differences in hindlimb motor scores between the single-dose and multiple-dose baicalein-treated groups on any testing day. Similarly, beam walk latencies were significantly shorter when compared to those of the vehicle group on days 4 and 21 for the single-dose baicalein group and on days 1, 4, 7, 21 and 28 for the multiple-dose group. The beam walk latencies were significantly shorter for the multiple-dose baicalein-treated group than those for the single-dose group on days 1 and 4. Values are means±s.e.m.; *P<0.05, **P<0.01, ***P<0.001 versus vehicle-treated injured rats. ^{†}P<0.05, ^{†\}P<0.01, single-dose versus multiple-dose baicalein-treated injured rats (n=8 for each group).

FIGS. 2 A to B show the effects of single-dose and multiple-dose baicalein treatment on cortical contusion volume, as evaluated by cresyl violet staining, on the fourteenth and twenty-eighth days post-injury. FIG. 2A represents cresyl violet-stained brain sections of a vehicle- and single-dose baicalein-treated injured rat, 14 days post-injury showing hypointensive regions and an obvious cavitation immediately below the impact site (*) in the cortex. Scale bar is 1 mm. As shown in FIG. 2B, bar graphs demonstrate cortical contusion volumes in vehicle-treated and baicalein-treated injured rats on days 14 and 28. Both single-dose and multiple-dose baicalein-treated rats had a significant reduction in contusion volume relative to vehicle-treated rats on days 14 and 28. Values are means±s.e.m.; *P<0.05 versus vehicle-treated injured rats (n=8 for each group).

FIGS. 3A to E show the effect of single-dose baicalein treatment on neuronal degeneration in rats with controlled cortical impact (CCI) injury, as evaluated by FluororJade B (FJB) staining 1 day post-injury. FIG. 3A shows a representative FJB-stained brain section of a vehicle treated, injured rat on day 1 postinjury. Scale bar is 1 mm. FIGS. 3B to 3D show high-power views of FJB-stained regions of interest on day 1 post-injury in a sham-injured control rat as shown in FIG. 3B, a baicalein-treated brain-injured rat as shown in FIG. 3C and a vehicle -treated brain-injured rat as shown in FIG. 3D. Note that there was a marked decrease in the number of FJB-positive cells after baicalein treatment. Scale bar is 100 mm. FIG. 3E shows bar graphs of mean densities of FJB-positive cells in vehicle-treated and baicalein-treated injured rats in the cortical contusion margin 1 day after injury showing a significant decrease in the number of FJB-positive cells in the baicalein-treated group. The total number of FJB-positive cells was expressed as the mean number per field of view (1.43 mm²). Values are means±s.e.m.; **P<0.05 versus vehicle-treated injured rats (n=7 for each group).

FIGS. 4A to 4B show the expression of inflammatory cytokine mRNAs after controlled cortical impact (CCI) injury, assessed by Taqman RT-PCR. FIG. 4A shows expression of tumor necrosis factor (TNF)-α, interleukin (IL)-1β, and IL-6 mRNAs increased significantly in the injured hemisphere compared with the corresponding region of the sham-injured controls at 3 and 6 h. FIG. 4B shows bar graphs demonstrating TNF-α, IL-1β and IL-6 mRNA expressions in sham-control, vehicle and single-dose baicalein-treated injured rats in ipsilateral cortices 6 h post-injury. Single-dose baicalein significantly reduced injury-induced TNF-α, IL-1β and IL-6 mRNA expressions in the ipsilateral hemisphere compared with vehicle-treated rats. Values are means±s.e.m.; *P<0.05, **P<0.01, ***P<0.001 versus sham-control and ^{†}P<0.05, ^{††}P<0.01, baicalein versus vehicle-treated injured rats (n=7 for each group).

FIGS. 5A to 5B show concentrations of inflammatory cytokine proteins after controlled cortical impact (CCI) injury, assessed by ELISA. FIG. 5A shows tumor necrosis factor (TNF)-α, interleukin (IL)-1β and IL-6 protein levels in untreated, injured rats significantly increased in the ipsilateral cortex from 6 to 96 h and peaked at 1 day. FIG. 5B shows bar graphs of TNF-α, IL-1β and IL-6 protein concentrations in sham control, vehicle and single-dose baicalein-treated injured rats in ipsilateral cortices 1 day post-injury. Baicalein significantly reduced the injury-induced increases of TNF-α, IL-1β and IL-6 protein concentrations compared with vehicle-treated injured rats. Values are means±s.e.m.; *P<0.05, **P<0.01, ***P<0.001 versus shamcontrol and ^{†}P<0.05, ^{††}P<0.01, baicalein versus vehicle-treated injured rats (n=7 for each group).

FIGS. 6A to 6B shows the effects of single-dose baicalein treatment on cytokine protein expression in rats with controlled cortical impact (CCI) injury, as evaluated by cytokine immunoreactivity 1 day post-injury. FIG. 6A shows a representative cresyl violet-stained brain section of a vehicle-treated injured rat 1 day post-injury showing the region of interest. Scale bar is 1 mm. FIG. 6B shows high-power photomicrographs of the regions of interest showing tumor necrosis factor (TNF)-α, interleukin (IL)-1β and IL-6 immunoreactivity 1 day post-injury in the sham-injured control, vehicle and single-dose baicalein-treated injured animals. Note that there was a marked rise in the number of TNF-α-, IL-1β- and IL-6-positive cells (arrows) after injury. The number of TNF-a-positive, IL-1b- and IL-6-positive cells was significantly reduced in baicalein-treated animals compared with vehicle-treated animals. Scale bar is 30 mm for TNF-α, IL-1β and IL-6 immunostaining.

FIGS. 7A to 7C show mean densities of immunopositive profiles displaying tumor necrosis factor (TNF)-α as shown in FIG. 7A, interleukin (IL)-1β as shown in FIG. 7B and IL-6 immunoreactivity as shown in FIG. 7C in vehicle-treated and single-dose baicalein-treated injured rats in the cortical contusion margin 1 day post-injury. There was a significant decrease in the number of both TNF-α-positive, IL-1β- and IL-6-positive cells in baicalein-treated rats. The total number of TNF-α-, IL-1β- and IL-6-positive cells was expressed as the mean number per field of view (2.56 mm²). Values are means±s.e.m.; *P<0.05 versus vehicle-treated injured rats (n=7 for each group).

FIG. 8 shows the chemical formula of Baicalein in the prior art.

### DETAILED DESCRIPTION OF THE PREFERRED EMBODIMENT

Accordingly, the invention provides a novel medical use of baicalein, and in particular, to the use of baicalein in the preparation of a pharmaceutical composition useful for treating traumatic brain injury.

In which, baicalein is useful for treating traumatic brain injury, and in particular, baicalein or a pharmaceutical composition containing the same is administrated immediately after traumatic brain injury to reduce the volume of traumatic brain injury and improve the Behaviour function deficit caused by traumatic brain injury.

The term “the volume of traumatic brain injury” refers to the area of the traumatic brain injury region calculated with analytical software, and to the contusion volume calculated with following formula:

Contusion volume=contusion area of each slice×thickness of each slice.

Term “Behaviour function deficit” is estimated in the invention based on following Behaviour test model: (a) rotarod, (b) tactile adhesive removal, (c) modified neurological severity score (mNSS) and (d) beam walk tests, and is not intended to limit the practical scope of the invention. Said Behaviour function deficit includes various types of Behaviour abnormality, un-coordination, deficit and the like induced by traumatic brain injury.

Further, the administration of baicalein or a pharmaceutical composition containing the same immediately after traumatic brain injury can reduce the biosynthesis of proinflammatory cytokines induced by traumatic brain injury. The term “biosynthesis” is meant to indicate the gene expression quantity of said cytokines which may be the expression quantity of mRNA or protein. The term “proinflammatory cytokines” is intended to include tumor necrosis factor-α (TNF-α), interleukin-1β (IL-1β), interleukin-6 (IL-6) or other relative inflammation factor.

Furthermore, the administration of baicalein or a pharmaceutical composition containing the same immediately after traumatic brain injury can improve secondary injury such as brain neuronal degeneration and the like in traumatic brain injury. The term “secondary injury such as brain neuronal degeneration and the like” means that, after traumatic brain injury, most of nerve cells in the region surrounding the injury tend to apoptosis and hence increase the extent of brain injury. Therefore, the term “to improve secondary injury such as brain neuronal degeneration and the like in traumatic brain injury” means to retard the apoptosis of neural cell.

In addition to the novel medical use of baicalein or a pharmaceutical composition containing the same in the treatment of traumatic brain injury, the invention further provides a pharmaceutical composition containing baicalein for treating traumatic brain injury or the secondary injury thereof.

The invention further provides a baicalein pharmaceutical composition useful for treating traumatic brain injury, wherein said pharmaceutical composition comprises a treating effective amount of baicalein and pharmaceutically acceptable excipient or carrier suitable for the use of said baicalein preparation. For example, when the excipient is solid, said baicalein pharmaceutical composition may be a capsule or a tablet. When said excipient is a vehicle, said baicalein pharmaceutical composition may be a liquid baicalein preparation. In following preferred examples, said liquid baicalein preparation is a baicalein injection solution. The dosage of baicalein injected in a rat is 30 mg baicalein per kilogram of body weight; however, said dosage is not intended to limit the dosage range practicable in the invention. Further, the administration route applicable in the invention includes, but is not limited to, single-dose administration, multiple-dose administration or multiple-sub-dose administration.

The excipient that can be used in the invention comprises, but is not limited to, diluent, filler, binder, disintegrating agent, lubricant and the like. Further, said excipient includes, but not limited to microcrystalline cellulose, polyvinylpyrrolidone (PVP), corn starch, modified starches, sodium carboxymethylstarch, resin, gelatinized starches, sugars, polyethylene glycol (PEG), polyvinyl alcohol, hydroxypropyl cellulose, methyl cellulose, hydroxymethyl cellulose, hydroxypropyl methyl cellulose and the like.

The term “treating effective amount” refers to the amount of a compound or a combination of compounds used to treat disease, to improve, attenuate or eliminate one or more symptoms of a particular disease, or to prevent or delay the outbreak of one or more symptoms caused by traumatic brain injury.

The term “pharmaceutically acceptable” is intended to mean that a substance or a combination has to be compatible with other components in the same formulation, and also has to be not harmful or cause no other side effect to a patient.

The novel medical use of baicalein provided by the invention comprises not only administration immediately after traumatic brain injury, but also routine taking or administration, so as to retard, protect or treat various symptoms caused by traumatic brain injury, thereby achieve the purpose of heath protection, prevention and treatment of symptoms caused by various extent of traumatic brain injury in daily life.

The invention will be illustrated with the examples as follows, without the intention that the invention is limited thereto.

### EXAMPLE 1

### Materials and Methods

### 1. Preparation of Baicalein

Baicalein was synthesized as described earlier (Huang et al., 2003). Briefly, the mixture of equimolar (20 mmol) trimethoxyphenol and cinnamoyl chloride was converted, through the Fries reaction in the presence of boron trifluoride-etherate, to the corresponding trimethoxychalcone. Further oxidation and cyclization of trimethoxylchalcone by catalytic iodine in dimethyl sulphoxide (Merck, Darmstadt, Germany) gave a crude trimethoxyflavone, followed by demethylation using hydrobromic acid and acetic acid. Final recrystallization was from hexane/ethyl acetate. The synthetic baicalein was further purified by column chromatography with silica gel in acetone/hexane (9/1). The purity was identified by HPLC through Shimadzu SPD-20A series instrument with a Purospher STAR RP-18e column (150×4.6 mm, 5 μm). The retention time of baicalein was at 2.146 min with a mobile phase of MeOH/water at isocratic elution at 1 mL min⁻¹. The purity of synthetic baicalein is over 98.5%, compared with commercial baicalein.

### 2. Surgical Procedures

All animals were treated in accordance with the International Guidelines for animal research, and the study design was approved by the animal ethics committee of Cheng Hsin Rehabilitation Medical Center. Animals were housed in groups in a temperature- (21-25° C.) and humidity (45-50%)-controlled room with a 12 hours light/dark cycle and ad libitum access to pellet chow and water.

A previously described CCI injury procedure was utilized (Chen et al., 2003). Male Sprague-Dawley rats, which have body weight of 250-300 g, were anaesthetized with sodium pentobarbital (i.p.; 50 mgkg⁻¹; Rhone Merieux, Harlow, UK) and placed in a stereotaxic frame. A 5-mm craniotomy was performed over the left parietal cortex, centered on the coronal suture and 3 mm lateral to the sagittal suture. Considerable care was taken to avoid injury to the underlying dura. Injury was made using a pneumatic piston with a rounded metal tip (2.5 mm diameter) that was angled 22.5° C. to the vertical so that the tip was perpendicular with the brain surface at the centre of the craniotomy. A velocity of 4 ms⁻¹ and a deformation depth 2 mm below the dura were used. The bone flap was immediately replaced and sealed, and the scalp was closed with sutures. Body temperature was monitored throughout the surgery by a rectal probe; temperature was maintained at 37.0±0.5° C. using a heated pad. Rats were placed in a heated cage to maintain body temperature, while recovering from anaesthesia.

Sham-operated rats received a craniotomy as before but without CCI; the impact tip was placed lightly on the dura before sealing the wound. After the trauma or sham surgery, animals were housed under the same conditions, as described above.

### 3. Experimental Protocol

### Study 1.

Baicalein (30 mg kg⁻¹) dissolved in 10% dimethyl sulphoxide (0.25-0.3 mL) or a corresponding volume of vehicle (10% dimethyl sulphoxide) was administered i.p. immediately following injury or daily for 4 days (immediately, 25, 49 and 73 h) after injury. Behaviour testing using the rotarod, tactile adhesive removal, mNSS and beam walk tests was performed at 28 hours as well as 4, 7, 14, 21 and 28 days post-injury. At 14 and 28 days, the animals were killed, perfused intravascularly and their brains were processed with cresyl violet staining to assess contusion volume (n=8 for each group). The dose of baicalein administration was selected based on pilot studies conducted in our laboratory in which 10 and 30 mg kg⁻¹ were tested and 30 mg kg⁻¹ was found to have neuroprotetcive effects in improving behavioural deficits (unpublished observations).

### Study 2.

The temporal profile of cytokine mRNA and protein levels was evaluated in a further group of injured and sham rats by RT-PCR and ELISA analysis. The purpose was to determine the optimal time point to examine the effect of baicalein on TNF-α, IL-1β and IL-6 mRNA and protein expression. Following the CCI procedure, rats were processed for RT-PCR at 3, 6, 18 and 24 hours and ELISA at 3, 6, 24 and 96 hours (all times post-injury; n=7 for each time point). Fourteen additional sham-operated rats were used for RT-PCR and ELISA analysis (n=7 for each group).

### Study 3.

Single-dose baicalein (30 mg kg⁻¹) or a corresponding volume of vehicle was administered i.p. immediately following injury. The single-dose regimen was chosen from the results of study 1 (in which single-dose baicalein significantly improved neurological outcomes) and study 2 (in which cytokine expression peaked early post-injury; see Results). Testing after injury was as follows: (1) real-time quantitative RT-PCR analysis for TNF-α, IL-1β and IL-6 mRNA expression at 6 h post-injury (n=7 for each group); (2) FluoroJade B (FJB) staining, TNF-α, IL-1β and IL-6 immunohistochemistry, as well as ELISA at 24 h post-injury to assess degenerative neurons and to determine cytokine expression (n=7 for each group). Twenty-one additional sham-operated rats were used for RT-PCR, ELISA and histology analysis (n=7 for each group).

### 4. Neurological Function Evaluation

Behaviour testing was performed before CCI and at 1, 4, 7, 14, 21 and 14 days after CCI by an observer who was unaware of the experimental treatments. The battery of tests consisted of the rotarod motor test, tactile adhesive-removal somatosensory test, mNSS and beam walk test. Animals were pretrained for 3 days for the rotarod, tactile adhesiveremoval somatosensory and beam walk tests (see below).

### 5. Rotarod Test

An accelerating rotarod was used to measure rat motor function and balance (Hamm, 2001). Each rat was placed on the rotarod cylinder, and the time for which the animal remained on the rotarod was measured. Speed was slowly increased from 4 to 20 r.p.m. within 5 min. A trial would be ended if the animal fell off the rungs or gripped the device and spun around for two consecutive revolutions without attempting to walk on the rungs. One hour before CCI, the mean duration on the device was recorded with three rotarod measurements as the pre-injury baseline values. Post-injury latencies were expressed as a percentage of their respective baseline values to reduce inter-animal variability.

### 6. Tactile Adhesive-Removal Test

For the tactile adhesive-removal somatosensory test, two small adhesive-backed paper dots (each 113.1 mm²) were used as bilateral tactile stimuli occupying the distal-radial region on the wrist of each forelimb (Chen et al., 2001). The time required for each rat to remove adhesive from the forelimb was recorded for five trials per day. Individual trials were separated by at least 5 min. One hour before CCI, mean latency (in seconds) over five trials to remove the contralateral (right) adhesive was recorded as the pre-injury baseline value.

### 7. Modified Neurological Severity Score

As shown in Table 1, the mNSS is a composite of motor, sensory, reflex and balance tests (Chen et al., 2001). One point was scored for the inability to perform the test or for the lack of a tested reflex; thus, the higher the score, the more severe the injury. Neurological function was graded on a scale of 0-18 (normal score, 0; maximal deficit score, 18).

TABLE 1
Modified neurological severity scores (mNSS)

| Tests | Point |
|---|---|
| Motor tests | |
| Raising the rat by the tail | |
| Flexion of forelimb | 1 |
| Flexion of hindlimb | 1 |
| Head moving more than 10° (vertical axis) | 1 |
| Placing the rat on the floor | |
| Inability to walk straight | 1 |
| Circling towards the paretic side | 1 |
| Falling down to the paretic side | 1 |
| Sensory tests | |
| Visual and tactile placing | 1 |
| Proprioceptive test (deep sensory) | 1 |
| Beam balance tests | |
| Grasps side of the beam | 1 |
| Hugs the beam and one limb falls down from the beam | 2 |
| Hugs the beam and two limbs fall down from the beam or | 3 |
| spins on beam (>60 s) | |
| Attempts to balance on the beam but falls off (>40 s) | 4 |
| Attempts to balance on the beam but falls off (>20 s) | 5 |
| Falls off: no attempt to balance or hang on to the beam (<20 s) | 6 |
| Reflexes (blunt or sharp stimulation) absence of: | |
| Pinna reflex (a head shake when touching the auditory meatus) | 1 |
| Corneal reflex (an eye blink when lightly touching the cornea | 1 |
| with cotton) | |
| Startle reflex (a motor response to a brief loud paper noise) | 1 |
| Seizures, myoclonus, myodystony | 1 |
| Maximum points | 18 |

One point is awarded for the inability to perform the tasks or for the lack of a tested reflex.

### 8. Beam Walk Test

The beam walk test was utilized to evaluate fine motor coordination and function (Feeney et al., 1982). Rats escaped from a bright light and loud white noise by walking along a narrowed wooden beam (2.5×122.0 cm) to enter a darkened goal box at the opposite end of the beam. The latency for the rat to reach the goal box (not to exceed 60 s) and hindlimb performance as he traversed the beam (based on a 1-7 rating scale) were recorded. A score of 7 was given when animals traversed the beam with two or less foot slips. A score of 6 was given when animals traversed the beam with less than 50% foot slips. A score of 5 was given for more than 50% but less than 100% foot slips. A score of 4 was given for 100% foot slips. A score of 3 was given for traversal with the affected limb extended and not reaching the surface of the beam. A score of 2 was given when the animal was able to balance on the beam but not traverse it. A score of 1 was given when the animal could not balance on the beam. Three trials were recorded 1 hour before CCI (baseline), and each day after CCI. Mean values of latency and score for each day were computed.

### 9. Real-Time Quantitative RT-PCR

Brains from injured or sham animals were removed without fixation after cervical dislocation, 6 hours following injury or sham surgery. A 3-mm coronal section was taken from the injured area over the parietal cortex, snap-frozen in liquid nitrogen, and stored at −70° C. until use. Approximately 50 mg tissue was collected from the ipsilateral (injured) hemisphere and processed for real-time quantitative RT-PCR. Total RNA was extracted from tissue samples using the RNeasy Mini Kits manufactured by Qiagen located at Valencia, Calif., USA. The purity and quality of extracted total RNA were confirmed by determining the ratio of absorbance at 260 nm to that at 280 nm and by ethidium bromide staining of 1 mg total RNA separated on a 1.5% agarose gel. Nondegraded RNA shows well-defined bands for both the 18S and 28S RNAs with no visible degradation. One microgram total RNA was first treated with DNase I, then subjected to reverse transcription using SUPERSCRIPT II RNase H Reverse Transcriptase manufactured by Invitrogen located at Carlsbad, Calif., USA, following the manufacturer's protocol. Real-time quantitative RT-PCR analysis was performed with an ABI PRISM 7500 sequence detector (Applied Biosystems, Foster City, Calif., USA). The primers and probe for TNF-A (TaqMan Gene Expression Assay ID Rn00562055_g1), IL-1β (TaqMan Gene Expression Assay ID Rn000580432_g1) and IL-6 (TaqMan Gene Expression Assay ID Rn99999011_m1) were obtained from Applied Biosystems. The endogenous control was β-actin (TaqMan Gene Expression Assay ID Rn00667869_m1). FAM (6-carboxyfluorescin) was used as the reporter dye, with TAMRA (6-carboxytetramethyl-rhodamine) used as the quencher dye. Thermal cycling was initiated with a 2-minutes incubation at 50° C., followed by a first denaturation step of 10 min at 95° C. and then 40 cycles of 95° C. for 15 s and 60 1 C for 1 min. The ABI PRISM 7500 sequence detector measures fluorescence emission synchronized with the thermal cycler during each extension step. Each sample was run in triplicate. Relative standard curves for the candidate gene and β-actin ribosomal RNAs (rRNAs) were performed each time genes were analyzed. The value of β-actin rRNA, which was consistent regardless of experimental condition, was used for an internal control. All PCR products were analyzed in the geometric range of the exponential phase during PCR amplification. Relative quantities of the candidate genes and β-actin rRNA were calculated by the comparative threshold cycle (Cₜ) method. In brief, the Cₜ value of TNF-α, IL-1β or IL-6 gene was subtracted from the Cₜ value of β-actin rRNA (expressed as ΔCₜ) as a standard for the amount of RNA template and efficiencies of reverse transcription. Then the ΔCₜ of samples from injured rats or sham-control rats was normalized to the sham-control sample with the lowest ΔCₜ values. The resulting change in ΔCₜ values (expressed as ΔΔCₜ) was converted to a linear form using 2^{(-ΔΔCt)}; and the transformed value was used in subsequent statistical analysis.

### 10. ELISA

Brains from injured or sham-control animals were removed without fixation after cervical dislocation one day following surgery. A 3-mm coronal section was taken from the injured area over the parietal cortex, snap-frozen in liquid nitrogen, and stored at −70° C. until use. Brain samples were homogenized in a buffer consisting of 0.05M Tris-HCl, 0.15M NaCl, 0.1% Nonidet 40, 0.5M phenylmethylsuplhonyl fluoride, 50 μg mL⁻¹ aprotinin, 10 μg mL⁻¹ leupeptin, 50 μg mL⁻¹ pepstatin, 4 mM sodium orthovanadate, 10 mM sodium fluoride and 10 mM sodium pyrophosphate. Homogenates were centrifuged at 4° C. and 12000×g for 15 min. Supernatants were removed and assayed in duplicate using R&D TNF-a, IL-1b and IL-6 assay kits (R&D Systems, Minneapolis, Minn., USA), according to the manufacturer's guidelines. Tissue cytokine concentrations were expressed as picograms of antigen per milligram of protein.

### 11. Tissue Processing and Histology

Following with terminal anaesthesia, rats were killed by transcardial perfusion first with phosphate-buffered saline (PBS) and then with 4% paraformaldehyde and their tissues were processed on day 1 (for FJB staining and cytokine immunohistochemistry), day 14 (for cresyl violet histology) and day 28 (for cresyl violet histology), post-injury. All solutions were maintained at pH 7.4 and 4° C. Brains were removed and post-fixed in 4% paraformaldehyde overnight and transferred to PBS containing 30% sucrose and 0.1% sodium azide, manufactured by Sigma Chemical Co. located at St Louis, Mo., USA, for cryoprotection. Coronal sections were cut in a cryostat at 10 mm from the level of the olfactory bulbs to the visual cortex. Every 50^{th} slice was used for cresyl violet histology, FJB staining or immunohistochemistry. The distance between similarly stained sections within each group was thus 500 mm, with approximately 20 sections per group. The following primary antibodies and dilutions were used: (1) goat polyclonal anti-TNF-a (E-20) (Santa Cruz Biotechnology, Santa Cruz, Calif., USA; 1:50); (2) rabbit polyclonal anti-IL-1β (BioSource International, Camarillo, Calif., USA; 1:200); (3) goat polyclonal anti-IL-6 (Santa Cruz Biotechnology; 1:200); (4) mouse monoclonal anti-neuronal nuclei antigen (NeuN) (Chemicon, Temecula, Calif., USA; 1:400); (5) mouse monoclonal anti-OX42 (Serotec, Raleigh, N.C., USA; 1:200);(6) mouse monoclonal anti-glial fibrillary acidic protein (GFAP) (Dako, Carpenteria, Calif., USA; 1:200).

### 12. Immunohistochemistry

All sections were dried, rehydrated in PBS, fixed in 4% paraformaldehyde for 20 minutes and rinsed in PBS. Sections were quenched in a solution of 10% methanol/10% hydrogen peroxide in distilled water for 5 minutes before washing three times in Trizma (Sigma)-buffered saline (TBS). Sections were blocked for 60 minutes in TXTBS (TBS containing 0.2% Triton X-100; Sigma) with 3% normal goat serum (NGS; Dako) and incubated overnight at 4° C. in the relevant primary antibody (goat polyclonal anti-TNF-α, rabbit polyclonal anti-IL-1β or goat polyclonal anti-IL-6) in TXTBS containing 1% NGS. After three washes in TBS, sections were left in the appropriate biotinylated secondary antibody (biotinylated anti-goat IgG or biotinylated anti-rabbit IgG; Vector, Burlingame, Vt., USA) at a concentration of 1:200 in TBS with 1% NGS for 3 hours, followed by three washes in TBS. The primary antibody was visualized with diaminobenzidine using a streptavidin-biotinylated horseradish peroxidase complex kit (Dako) in 1% NGS in TBS for 2 hours followed by three washes in TBS and two washes in Trizma nonsaline. Sections were developed with diaminobenzidine in Trizma nonsaline containing 0.03% hydrogen peroxide, and excess stain was removed by washing in Trizma nonsaline three times. Nonspecific staining was investigated by omitting the primary antibody and was negative.

### 13. FJB Histochemistry

FluoroJade B is a polyanionic fluorescein derivative that sensitively and specifically binds to degenerating neurons, and staining was carried out using a published technique (Schmued and Hopkins, 2000), with some modification. Briefly, sections were first incubated in a solution of 1% NaOH in 80% ethanol for 5 minutes and then hydrated in graded ethanol (75, 50 and 25%; 5 minutes each) and distilled water. They were incubated in a solution of 0.06% potassium permanganate for 10 min, rinsed in distilled water for 2 min and incubated in a 0.0004% solution of FJB (Chemicon) for 30 min. The slides were washed and mounted on coverslips with Vecta-shield mounting medium (Vector). All sections were observed and photographed under a fluorescence microscope (Olympus BX-51) with blue (450-490 nm) excitation light.

### 14. Quantification of Cytokine and FJB Staining

Cytokine and FJB staining were quantified on TNF-α, IL-1β-, IL-6- and FJB-stained sections between Bregma level 0.2-0.6 mm. Three 10-mm sections per animal were randomized selected between the two levels and stained with FJB or cytokine stainings. The region of interest was defined and delineated under a 4× objective on each section as the TNF-α-, IL-1β-, IL-6- or FJB-positive cells in the contusion margin along the cortex. Using a ×20 objective, five randomly selected, non-overlapping fields with an area of 690 μm width and 520 μm height for cytokine immunostainings or an area of 1350 μm width and 1060 μm height for FJB stainings were examined. The total number of TNF-α-, IL-1β-, IL-6- and FJB-positive cells were expressed as the mean number per field of view. Analysis was conducted by two experimenters who were unaware of all animal characteristics. Inter-rater reliability in cell counts was well within 10%.

### 15. Contusion Volume

To measure contusion volumes, cresyl violet-stained sections were digitized and analyzed using a ×1.5 objective and computer image analysis system (Scion Image, Beta Release 4.0.2; Scion Corp., Frederick, Md., USA). Contusion volume measurement was performed as previously described (Chen et al., 2003). Contusion area was calculated from all images of cresyl violet-stained sections that contained contused brain; volume measurement was computed by summation of areas multiplied by interslice distance (500 μm).

### 16. Statistical Analyses

Data are presented as mean±s.e.mean. For ELISA measurement of cytokine levels, an overall difference between the groups were tested with one-way ANOVA and a post hoc (Bonferroni) test was used to determine individual group differences (n=7 for each group). Nonparametric Kruskall and Wallis rank analysis was used to evaluate the RT-PCR data with subsequent group comparisons using the Mann-Whitney U-test (n=7 for each group). For behaviour testing and contusion volume measurement, two-way ANOVA with repeated measurements followed by a post hoc (Bonferroni) test was used to determine significant differences (n=8 for each group). For TNF-α-, IL-1β-, IL-6- and FJB-positive cell counts, a Student t-test was used to determine significant differences (n=7 for each group). Differences between means were assessed at the probability level of P<0.05, 0.01, and 0.001.

### EXAMPLE 2

### Results

### 1. Body Weight

All animals lost a small proportion of body weight (˜6%) in the first 24 hours following CCI injury but regained baseline weight within 4 days. There was no significant difference between groups treated with baicalein or vehicle regarding body weight (P=0.8; data not shown).

### 2. Rotarod Test

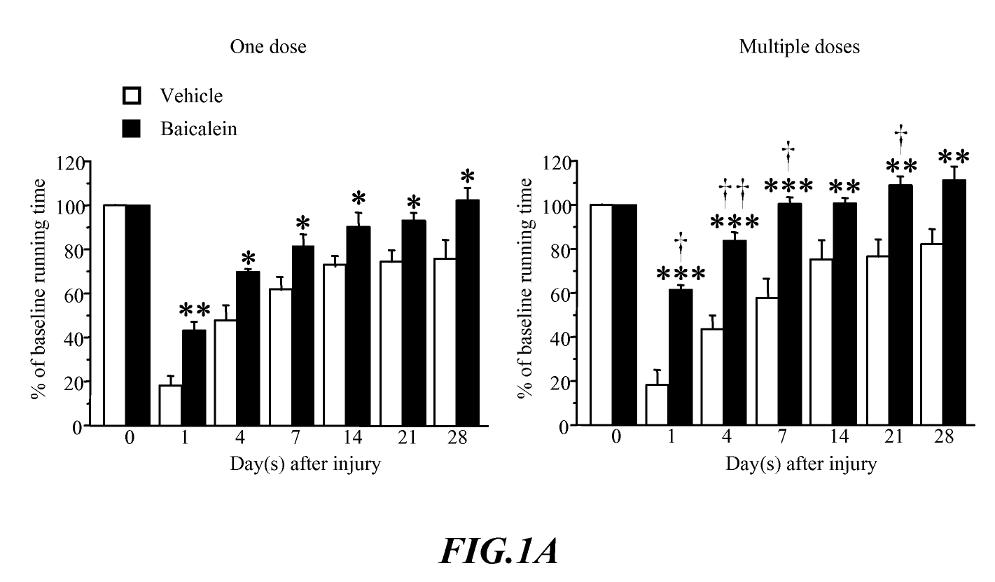

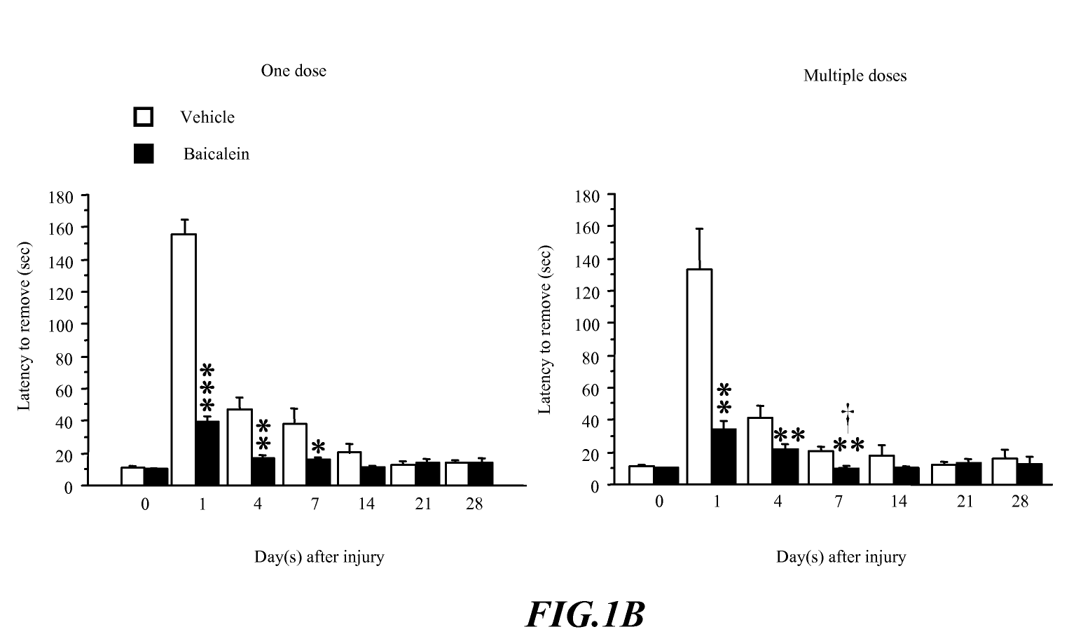

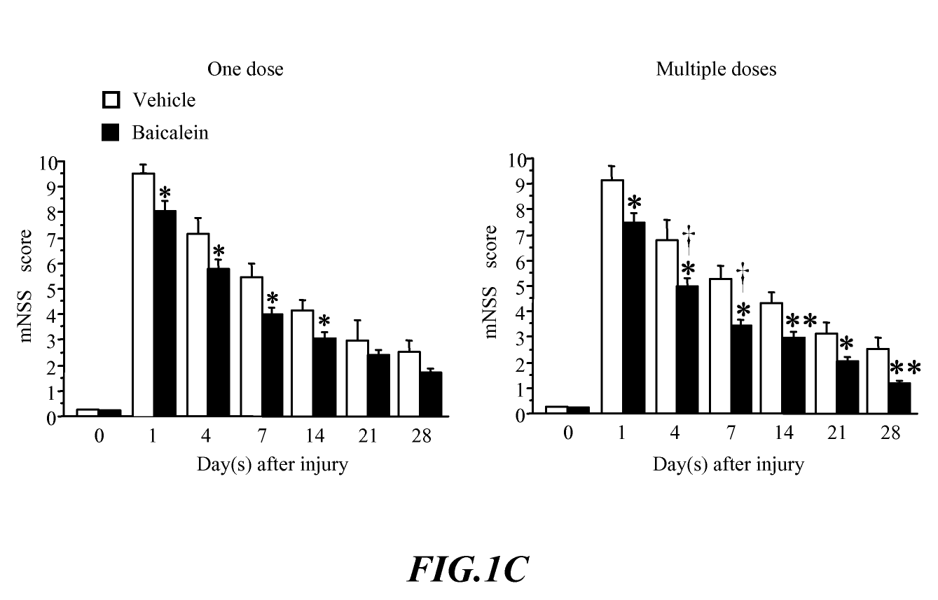

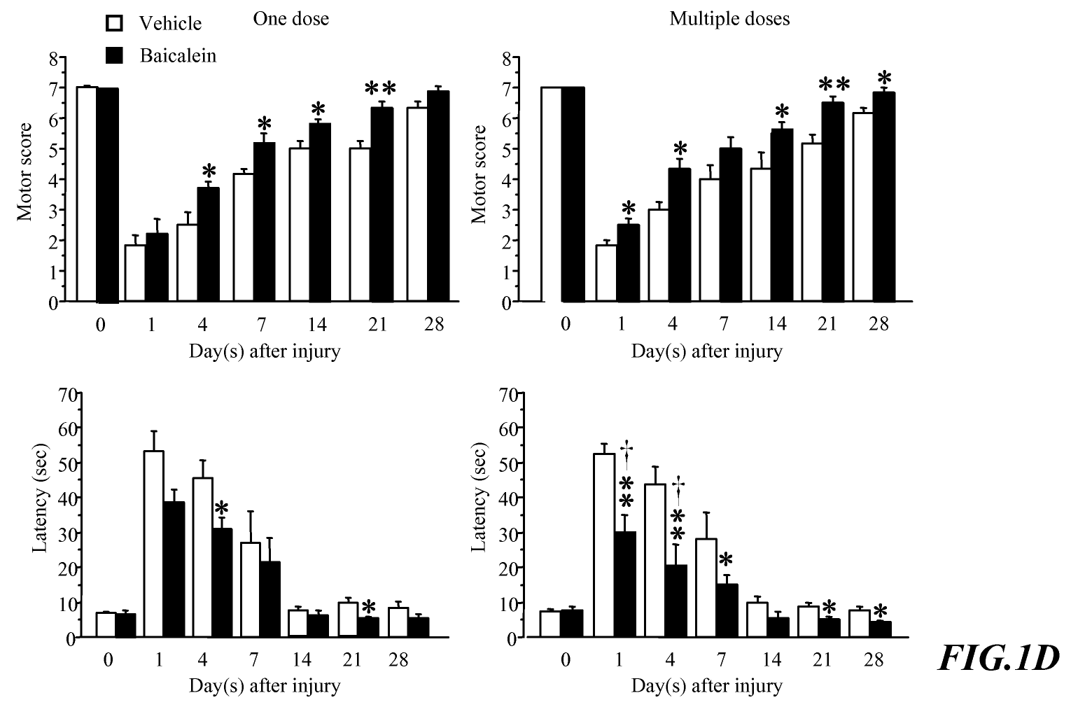

Motor function impairment caused by CCI was evident in the vehicle-treated group (FIG. 1a). Performance on the rotarod test was significantly better for both single-dose and multiple-dose baicalein-treated rats than for vehicle-treated rats on test days 1 to 28 after injury (all P<0.05) as shown in FIG. 1A. Multiple-dose treatment was significantly more effective than single-dose treatment on days 1 (P<0.05), 4 (P<0.01), 7 (P<0.05) and 21 (P<0.05) after injury.

It could be seen from the rotarod test that administration with four doses of 30 mg kg⁻¹ baicalein resulted better motor function restoration, however, since one dose of 30 mg kg⁻¹ baicalein could give a remarkable effect, the minimum effective dosage was used as the treating dosage in this example.

### 3. Tactile Adhesive Removal Test

In both baicalein-treated and vehicle-treated groups of rats, unilateral contusion resulted in a delay in the time needed to remove the patch as shown in FIG. 1B. Both the single-dose and multiple-dose baicalein-treated rats had significantly lower adhesive-removal times at 1, 4 and 7 days than vehicle-treated rats (all P<0.05) had. Rats treated with multiple doses of baicalein had significantly lower adhesive-removal times only on day 7 after injury, as compared with those treated with single dose of baicalein (P<0.05).

### 4. Modified Neurological Severity Score

Injury in the left hemispheric cortex resulted in neurological functional deficits as measured by mNSS as shown in FIG. 1C. The mNSS scores were significantly less for the single-dose baicalein-treated group than the corresponding vehicle-treated group on days 1-14 (all P<0.05) and significantly less for the multiple-dose group than the corresponding vehicle-treated group on days 1-28 (all P<0.05). As compared with single-dose treatment, multiple-dose treatment resulted in a significantly greater reduction of neurological deficit on days 4 and 7 after injury (both P<0.05).

### 5. Beam Walk Test

Marked impairment in beam walk performance was observed on the first day after surgery, regardless of treatment as shown in FIG. 1D. The decrease in hindlimb motor scores was significantly different between the single-dose group and vehicle group on days 4, 7, 14 and 21 (all P<0.05) and between the multiple-dose group and vehicle group on days 1, 4, 14, 21 and 28 (all P<0.05). However, differences in hindlimb motor scores between the single-dose and multiple-dose groups were not significant on any testing day (all P>0.05). Similarly, beam walk latencies were significantly shorter for the single-dose group than the vehicle group on days 4 and 21 (both P<0.05) and for the multiple-dose group than the vehicle group on days 1, 4, 7, 21 and 28 (all P<0.05). The beam walk latencies on days 1 and 4 were significantly shorter for the multiple-dose group than the single-dose group (both P<0.05).

### 6. Post-Injury Baicalein Treatment Reduces Neuronal Injury

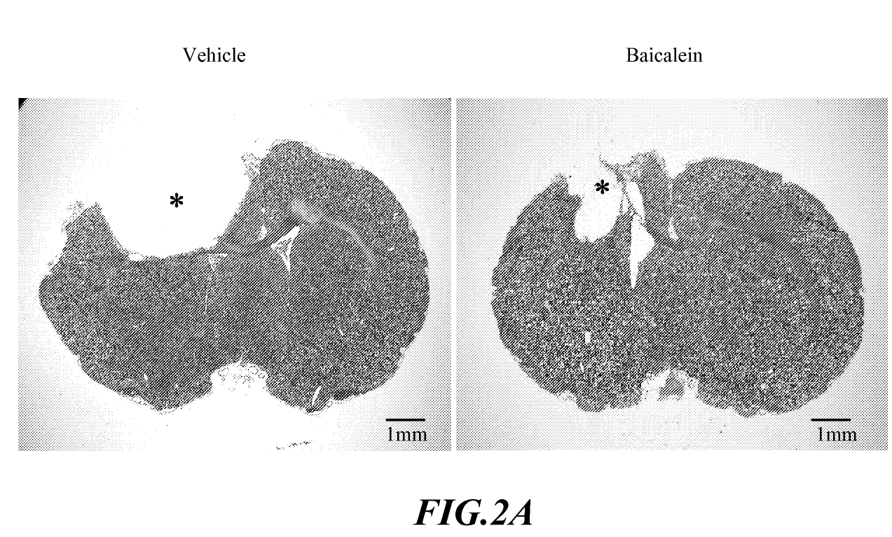

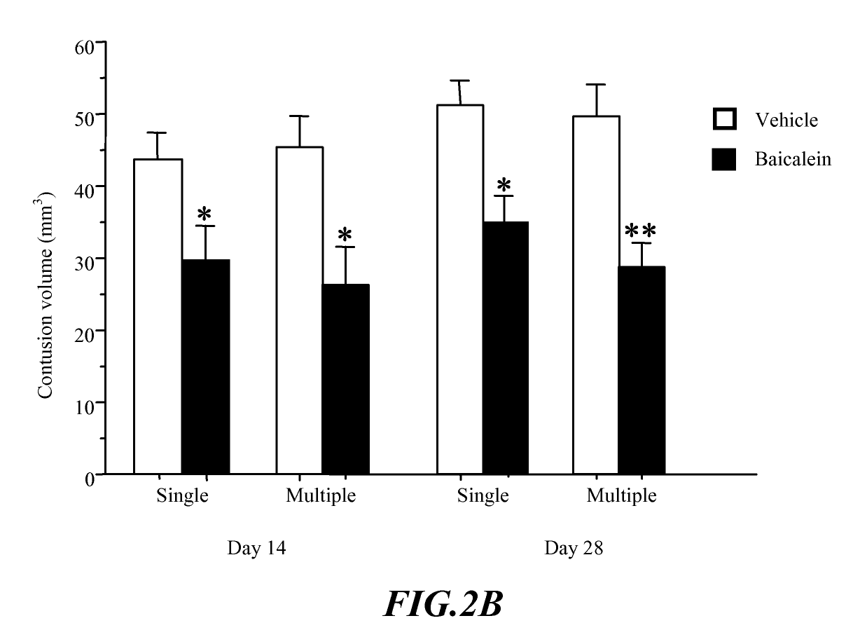

Controlled cortical impact injury resulted in a loss of cortical tissue in the ipsilateral parietal cortex, as reflected by gross reductions in cresyl violet staining intensity as shown in FIG. 2A. In contrast, the cytoarchitecture of the cortex remained normal in the contralateral hemisphere. On the fourteenth day, the volume of contusion estimated from cresyl violet-stained sections after vehicle treatment was the same for single-dose and for multiple-dose treatment, and these values were significantly greater than the contusion volume after baicalein treatment, either after a single dose or after multiple doses (both P<0.05; FIG. 2b). The contusion volume increased, up to 28 days post-injury, after single-dose and multiple-dose vehicle treatment (FIG. 2b) and this volume was significantly reduced after single-dose (P<0.05) or multiple-dose bacalein treatment (P<0.01; FIG. 2b). However, there was no significant difference in contusion volume between the single-dose and multiple-dose baicalein-treated groups on both testing days (both P>0.05). Overall, single and multiple doses of baicalein reduced contusion volume, respectively, by 32 and 42% at 14 days post-injury and by 34 and 42% at 28 days post-injury.

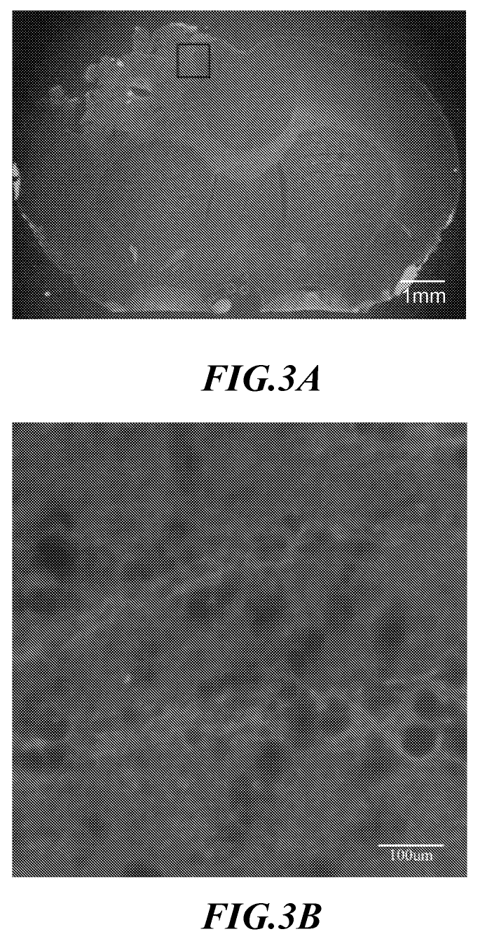

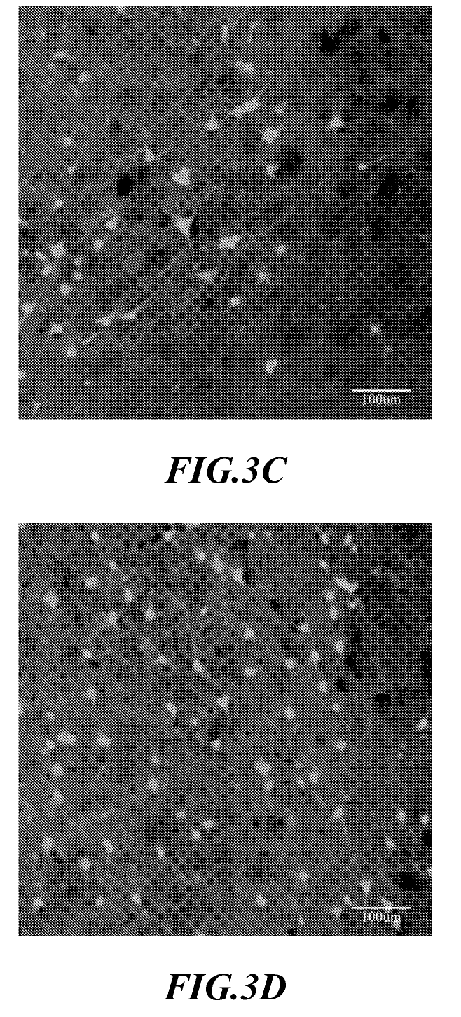

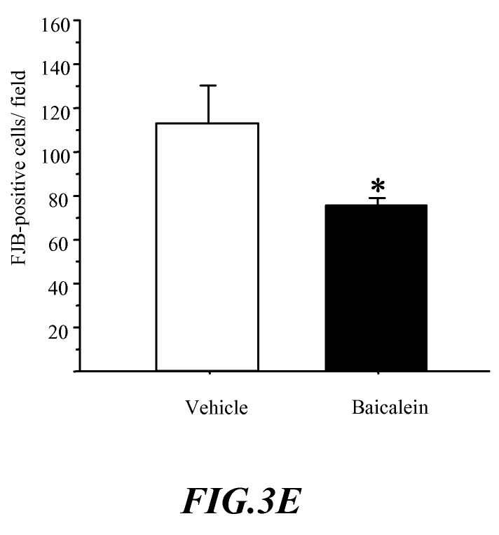

As FJB reactivity has been shown to be maximal 1 day after moderate CCI injury, the time point of 1 day post-injury was chosen for FJB staining in our model. FJB-positive cells with neuronal morphology were evident 1 day after injury in the cortical contusion margin (FIG. 3A) and striatum in the ipsilateral, but not the contralateral hemisphere. Single-dose baicalein significantly reduced (by 31%) the number of FJB-positive cells compared with vehicle treatment (FIG. 3B-3E; P<0.05).

7. Post-Injury Baicalein Treatment Downregulates Proinflammatory Cytokine mRNA Expression

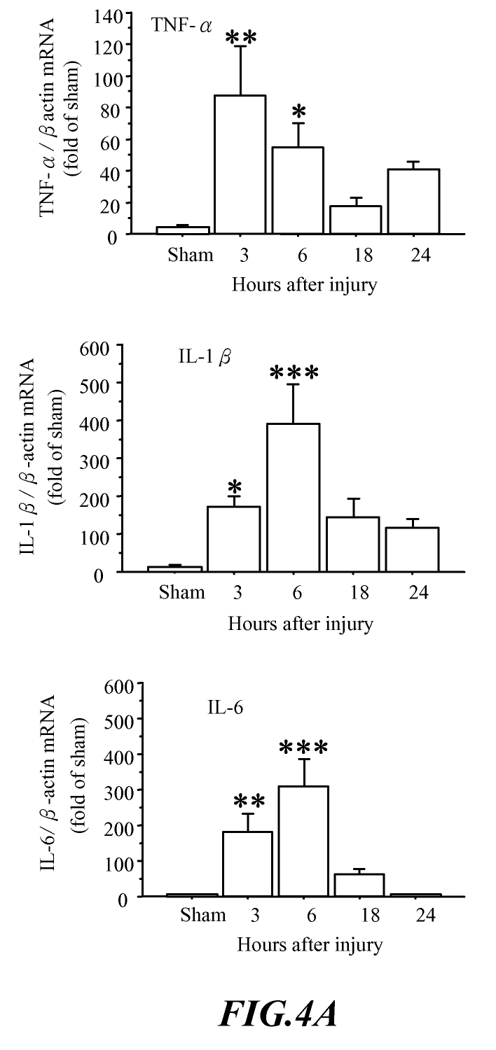

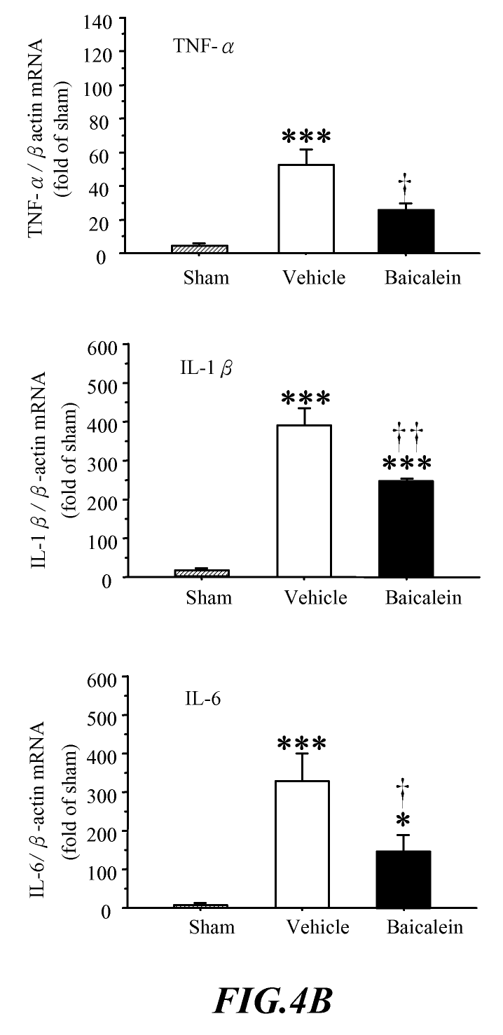

As single-dose baicalein treatment significantly improved neurological outcomes, we investigated whether this treatment paradigm reduced proinflammatory cytokine expression as hypothesized. Following injury, the mRNA expression increased significantly in the injured hemisphere for TNF-α, IL-1β and IL-6 compared with the corresponding region of sham controls at 3 and 6 hours (P<0.05 for all values) as shown in FIG. 4A. The peak level of TNF-α, IL-1β and IL-6 mRNA was observed around 6 hours; therefore, the time point of 6 hours was selected for evaluating treatment effect on cytokine mRNA expression. Vehicle-treated injured rats had a 14-fold higher TNF-α level, a 33-fold higher IL-1β level and a 60-fold higher IL-6 level than sham controls as shown in FIG. 4B. The profound increase in proinflammatory cytokines (associated with trauma) was significantly attenuated by baicalein treatment; as there was significant reduction in TNF-α, IL-1β and IL-6 mRNA levels 6 h after injury. On average, TNF-α, IL-1β and IL-6 mRNA levels in brains of baicalein-treated injured rats were 49% (P<0.05), 63% (P<0.01) and 43.6% (P<0.05), respectively, of the levels found in vehicle-treated injured rats.

### 8. Post-Injury Baicalein Treatment Reduces Cytokine Protein Expression

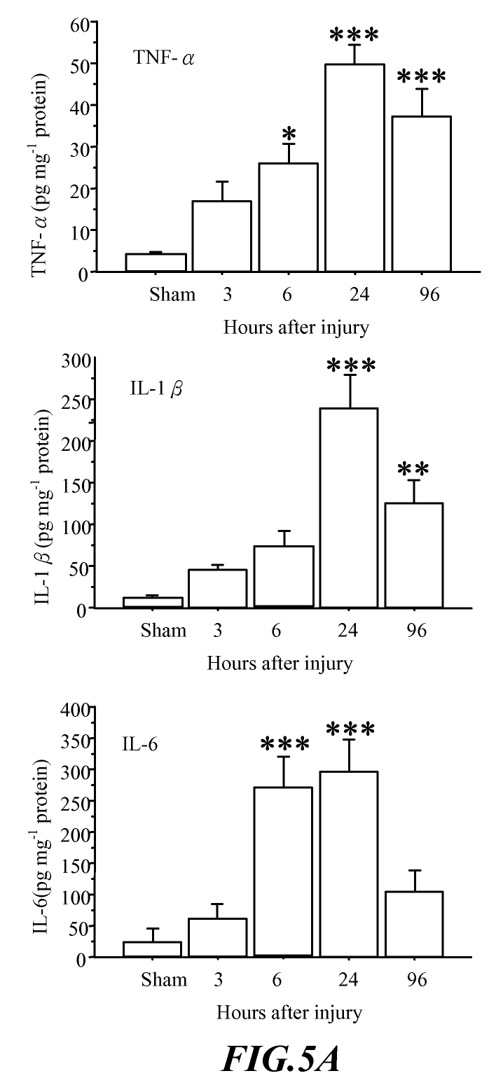

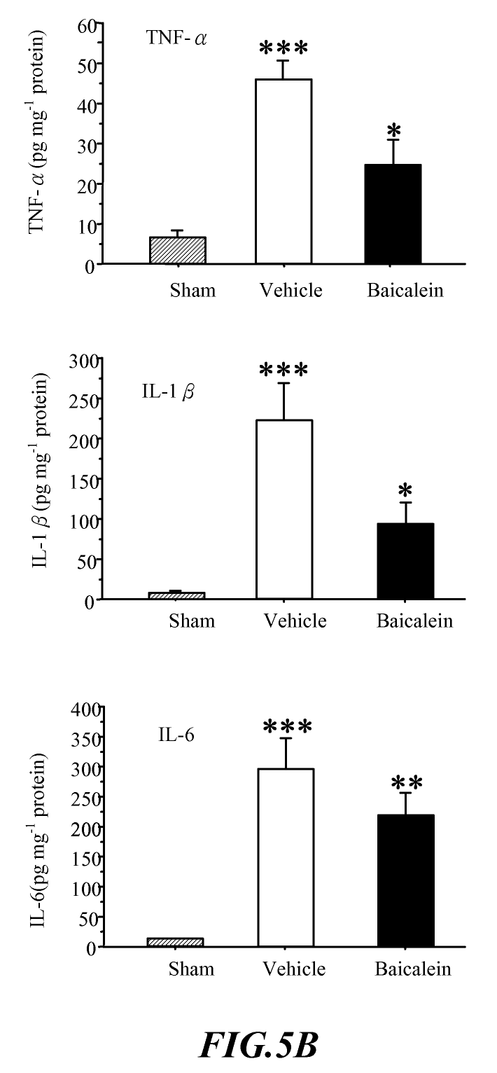

To examine the effect of baicalein on TNF-α, IL-1β and IL-6 protein expression, ELISA and immunohistochemistry were used to confirm gene translation one day after the injury. Basal protein levels of TNF-α, IL-1β and IL-6 were low in the cortex of sham-injured animals. After the injury, TNF-α, IL-1β and IL-6 protein levels in the ipsilateral cortex increased significantly from 6 to 96 hours and peaked on the first day as shown in FIG. 5A. Therefore, day 1 post-injury was selected for evaluating the effect of treatment on cytokine protein expression. At this time, cytokine levels in the ipsilateral cortex of vehicle-treated, injured rats were significantly increased; TNF-α, by 7-fold (P<0.001); IL-1β, by 30-fold (P<0.001) and IL-6 also by 30-fold (P<0.001), compared with sham values s shown in FIG. 5B). Baicalein significantly reduced the injury-induced increase in cortical tissue levels of all three cytokines as shown in FIG. 5B. On average, TNF-α, IL-1β and IL-6 protein concentrations in brains of baicalein-treated rats were 54% (P<0.05), 42% (P<0.01) and 67% (P<0.01), respectively, of the concentrations seen in vehicle-treated rats.

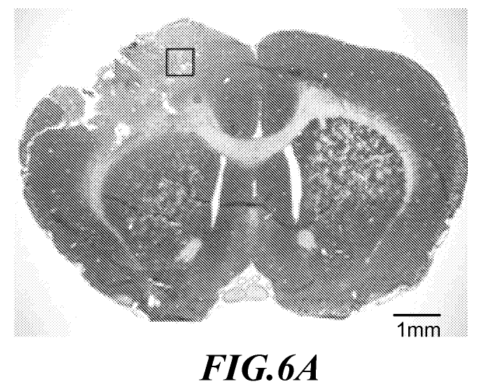

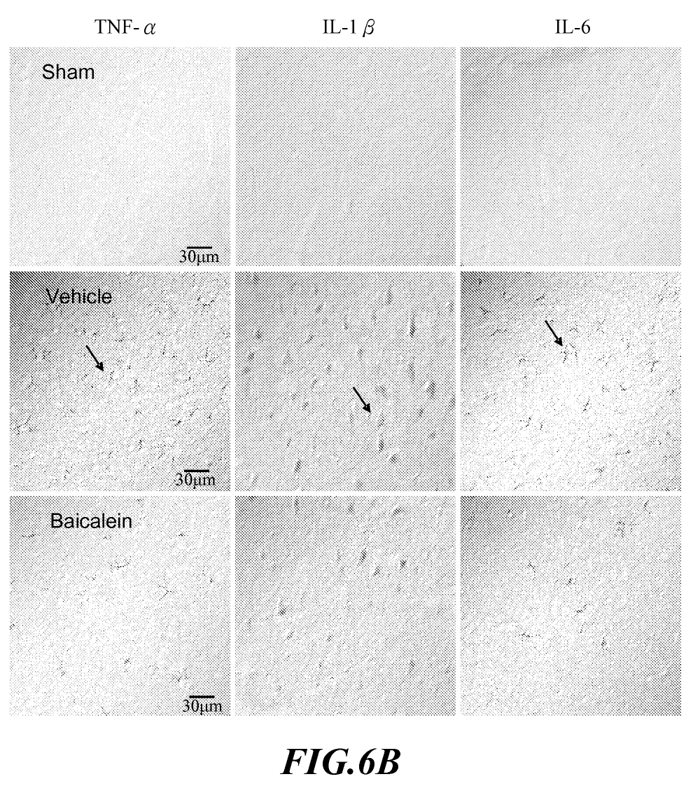

Cytokine immunohistochemistry indicated that neither normal brains nor contralateral hemispheres of injured brains displayed immunoreactivity for TNF-α, IL-1β or IL-6. However, TNF-α, IL-1β and IL-6 immunoreactivity was remarkably increased in the injured hemisphere by 1 day post-injury in vehicle-treated rats (FIGS. 6A-6B).

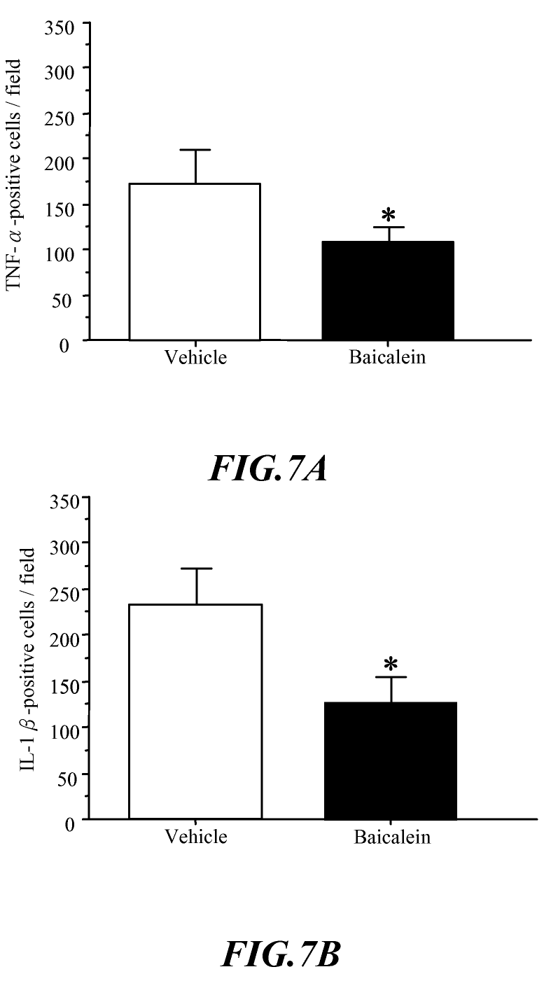

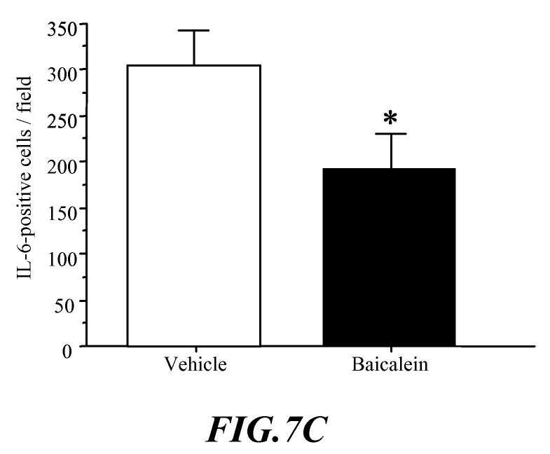

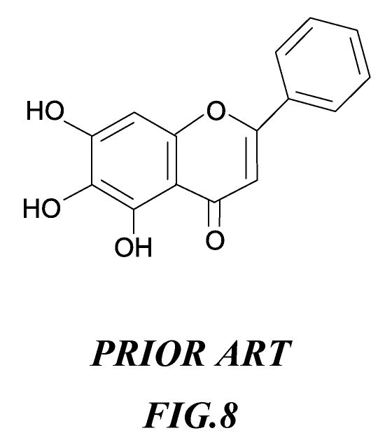

Baicalein significantly reduced the number of cells positive for TNF-α (P<0.01), IL-1β (P<0.05) and IL-6 (P<0.05) 1 day after trauma (FIGS. 6A-6B). Quantitative analysis revealed a 37% reduction in TNF-α-positive cells, a 40% reduction in IL-1β-positive cells and a 33% reduction in IL-6-positive cells, respectively, in cortical tissue adjacent to the contusion core in rats treated with baicalein relative to cell numbers in rats treated with vehicle (FIGS. 7A-7C).

### EXAMPLE 3

### Discussion

The invention show that post-injury administration of baicalein significantly reduced long-term neurological deficits and brain tissue damage following CCI. These effects correlate with a decrease of TNF-α, IL-β and IL-6 mRNA transcription and protein synthesis in the brain. The major novel finding in this invention is that baicalein, given i.p., improved both functional and histological outcomes in a model of CCI, perhaps through modulation of inflammation. This invention provides the first evidence that post-injury baicalein treatment can attenuate TBI-induced tissue damage and can improve functional recovery following TBI.

This invention suggests that baicalein may provide a potential therapy for TBI. Moreover, this invention suggest that the anti-inflammatory properties of baicalein contribute to its neuroprotective effect and provide further evidence that the post-traumatic inflammatory response contributes to the morphological and behavioural pathophysiology of TBI.

This invention shows that baicalein significantly improved long-term sensori-motor outcomes assessed using a combination of neurobehavioral tests. The reduction in functional deficits seen in the baicalein-treated animals correlated with the histological findings in this invention. Compared with vehicle-treated rats, baicalein-treated animals showed smaller contusion volumes and fewer degenerative neurons, as estimated by Nissl and FJB staining, respectively. Post-injury treatment with baicalein, therefore, protects some tissue that would otherwise be vulnerable to be damaged by trauma, resulting in amelioration of brain injury and improvement of functional outcome.

Also, the invention shows for the first time that post-TBI treatment with baicalein significantly reduced cytokine expression in parallel with reduced brain damage and neurological deficits. We have showed that baicalein suppressed the induction of proinflammatory cytokines in the injured brain following TBI. The invention shows that a single i.p. injection of baicalein, given immediately after the CCI procedure, had a protective effect for as long as 28 days (4 weeks) post-injury.

In conclusion, this invention indicates that post-injury treatment with baicalein leads to improve functional and histological outcomes in a clinically relevant model of TBI. This improvement was associated with attenuated expression of TNF-α, IL-1β and IL-6 mRNA and protein, suggesting that the neuroprotective effect of baicalein following TBI may be, in part, mediated through modulation of the injury-induced proinflammatory cascades. Among the advantages of baicalein treatment are that chronic dosing is not required, that baicalein has low levels of toxicity and that it is easy to administer in emergency situations. Thus, baicalein offers great promise as a potential treatment for TBI.

In summary, the novel medical use of Baicalein, and in particular, the use of Baicalein in the preparation of a pharmaceutical composition useful for treating traumatic brain injury provided by the invention is characterized in that it can improve the behaviour function deficit in traumatic brain injury, reduce the volume of traumatic brain injury, improve brain verve damage in traumatic brain injury, reduce proinflammatory cytokines such as tumor necrosis factor-α (TNF-α), interleukin-1β (IL-1β), interleukin-6 (IL-6) or other relative inflammatory factor induced in traumatic brain injury.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

What is claimed is:

1. Use of baicalein in the preparation of a pharmaceutical composition for the treatment of traumatic brain injury;
wherein said pharmaceutical composition comprises a treating effective amount of baicalein and suitable pharmaceutically acceptable excipient or carrier.

2. A use as recited in claim 1, wherein said baicalein is used to improve the behaviour function deficit in traumatic brain injury.

3. A use as recited in claim 1, wherein said baicalein is used to reduce the contusion volume of traumatic brain injury.

4. A use as recited in claim 1, wherein said baicalein is used to retard the brain neuronal degeneration in traumatic brain injury.

5. A use as recited in claim 1, wherein said baicalein is used to reduce proinflammatory cytokines in traumatic brain injury.

6. A use as recited in claim 5, wherein said proinflammatory cytokines is at least one selected from the group consisting of tumor necrosis factor-α (TNF-α), interleukin-1β (IL-1β), interleukin-6 (IL-6) and other relative inflammation factor.

7. A use as recited in claim 1, wherein when said carrier is a vehicle, said baicalein pharmaceutical composition is a liquid baicalein preparation.

8. A use as recited in claim 7, wherein said liquid baicalein preparation is a baicalein injection solution.

9. A use as recited in claim 1, wherein when said carrier is a solid, said baicalein preparation is a capsule.

10. A use as recited in claim 1, wherein when said carrier is a solid, said baicalein preparation is a tablet.
